SEQUENCE LISTING (11 sequences)

SEQ ID NO: 1 - DNA, 33 bp, Artificial Sequence
  Synthetic chBD2-F-NdeI
ggaattccatatgactacccctgtcccagtctc

SEQ ID NO: 2 - DNA, 37 bp, Artificial Sequence
  Synthetic chBD2-R-NdeI
cgatatccatatgaattacttgtccgtttatttctag

SEQ ID NO: 3 - DNA, 21 bp, Artificial Sequence
  Synthetic BamHI-NdeI
ggtaccgggggatcccatatg

SEQ ID NO: 4 - protein, 6 aa, Artificial Sequence
  Synthetic BamHI-NdeI
GTGGSH

SEQ ID NO: 5 - DNA, 60 bp, Artificial Sequence
  Synthetic AspHRV3CsBam-Sens
gtaccggtggttccgcgggtctggaagttctgttccaggggccctccgcgggttccggtg

SEQ ID NO: 6 - DNA, 60 bp, Artificial Sequence
  Synthetic AspHRV3CsBam-Anti
gatccaccggaacccgcggagggcccctggaacagaacttccagacccgcggaaccaccg

SEQ ID NO: 7 - protein, 22 aa, Artificial Sequence
  Synthetic polypeptide
GTGGSAGLEVLFQGPSAGSGGS

SEQ ID NO: 8 - DNA, 31 bp, Artificial Sequence
  Synthetic hCBDdelNdeIoligo-Sens
tcatcatcatcatcacatgaccactccggtg

SEQ ID NO: 9 - DNA, 31 bp, Artificial Sequence
  Synthetic hCBDdelNdeIoligo-Anti
caccggagtggtcatgtgatgatgatgatga

SEQ ID NO: 10 - protein, 101 aa, Pyrococcus furiosus
TTPVPVSGSLEVKVNDWGSGAEYDVTLNLDGQYDWTVKVKLAPGATVGSFWSANKQEGNGYVIFTPVSWNKGPTATFGFIVNGPQGDKVEEITLEINGQVI

SEQ ID NO: 11 - protein, 101 aa, Artificial Sequence
  Synthetic TN mutant of 258-358 positions of Pyrococcus furiosus chitinase
TTPVPVSGSLEVKVNDWGSGATYNVTLNLDGQYDWTVKVKLAPGATVGSFWSANKQEGNGYVIFTPVSWNKGPTATFGFIVNGPQGDKVEEITLEINGQVI

(19) United States
(12) Patent Application Publication
(10) Pub. No.: US 2015/0051373 A1
(43) Pub. Date: Feb. 19, 2015

(54) CELLULOSE-/CHITIN-TYPE POLYMERIC LIGHT-EMITTING MATERIAL

(71) Applicant: National Institute of Advanced Industrial Science and Technology, Chiyoda-ku, Tokyo (JP)

(72) Inventors: Hideto Hoshino, Ikeda-shi (JP); Koichi Uegaki, Ikeda-shi (JP)

(21) Appl. No.: 14/366,445

(22) PCT Filed: Nov. 21, 2012
(86) PCT No.: PCT/JP2012/080241

§ 371 Date: Jun. 18, 2014

(30) Foreign Application Priority Data
Dec. 19, 2011 (JP) 2011-277363
Jan. 27, 2012 (JP) 2012-014817

(51) Int. Cl. C07K 14/47

(52) U.S. Cl. CPC C07K 14/47; C07K 2319/60
USPC 530/350; 536/23.4

(57) ABSTRACT

The main object of the present invention is to provide a novel technique using a BAF. The invention that achieves the object provides the following: a chimeric protein comprising a luminescent domain and a cellulose- and/or chitin-binding domain, the luminescent domain comprising at least one luminescent protein selected from the group consisting of luciferases and fluorescent proteins.

### TECHNICAL FIELD

### Cross Reference to Related Applications

This application claims priority to Japanese Patent Application No. 2011-277363 filed on Dec. 19, 2011, and Japanese Patent Application No. 2012-014817 filed on Jan. 27, 2012, the entire contents of which are incorporated by reference herein.

The present invention relates to a chimeric protein capable of binding to cellulose and/or chitin, DNA encoding the chimeric protein or its complementary strand, and a luminescent material.

### BACKGROUND ART

Many polymer hydrolytic enzymes have a binding domain to a substrate. For example, cellulase and chitinase are known to have a cellulose-binding domain and a chitin-binding domain, respectively. Patent Literature 1 (JP4604185B) discloses a heat-resistant domain binding to both chitin and cellulose.

Patent Literature 2 discloses a fused protein of a fluorescent protein and a luciferase having high BRET (Bioluminescence Resonance Energy Transfer) efficiency based on the BAF (BRET-based Auto-illuminated Fluorescent-protein) technique.

### CITATION LIST

### Patent Literature

PTL 1: JP4604185B

PTL 2: JP2008-283959A

### SUMMARY OF INVENTION

### Technical Problem

An object of the present invention is to provide a novel technique that employs a luminescent domain.

### Solution to Problem

The present invention provides the below-described chimeric protein, which is capable of binding to cellulose and/or chitin, DNA that encodes the chimeric protein or its complementary strand, and a luminescent material.

Item 1: A chimeric protein comprising a luminescent domain and a cellulose- and/or chitin-binding domain, the luminescent domain comprising at least one luminescent protein selected from the group consisting of luciferases and fluorescent proteins.
Item 2: The chimeric protein according to Item 1, wherein the luminescent domain is bound to the cellulose- and/or chitin-binding domain directly or via a first linker.
Item 3: The chimeric protein according to either Item 1 or 2, wherein the luminescent domain comprises a luciferase and a fluorescent protein, and energy transfer (BRET) from the luciferase to the fluorescent protein can occur.
Item 4: The chimeric protein according to Item 3, wherein the luciferase is bound to the fluorescent protein via a second linker.
Item 5: The chimeric protein according to any one of Items 1 to 4, wherein the fluorescent protein is GFP, YFP, BFP, CFP, OFP, DsRED, or RFP.
Item 6: The chimeric protein according to Item 5, wherein the fluorescent protein is YFP or RFP.
Item 7: The chimeric protein according to any one of Items 1 to 6, wherein the first linker and/or the second linker comprises a protease cleavage sequence.
Item 8: DNA that encodes the chimeric protein according to any one of Items 1 to 7, or its complementary strand.
Item 9: A luminescent material wherein the chimeric protein according to any one of Items 1 to 7 is bound to a cellulose- or chitin-comprising granule, bead, sheet, or film.

### Advantageous Effects of Invention

The chimeric protein of the present invention, when bound to a granule, a bead, a sheet, a film, or the like, of a biopolymeric material, such as cellulose or chitin, and dried, can maintain its activity for a long period of time. Because luminescent proteins, when dried, are typically deactivated, the chimeric protein of the present invention is excellent as a luminescent material.

### BRIEF DESCRIPTION OF DRAWINGS

FIG. 1 shows the procedure for examining the drying resistance (room temperature storage) of a paper sample to which a chimeric protein has been applied and bound, followed by air-drying. The steps are explained below.

(A) A chimeric protein aqueous solution is added dropwise to a punched round filter paper sample to bind a chimeric protein to the filter paper.

(a-1) The filter paper sample is dried.

(a-2) The filter paper sample is stored at room temperature.

(B) A buffer is added thereto.

(b) This moistens the filter paper sample.

(C) A luciferin aqueous solution is added to the buffer containing the filter paper sample.

(c) The luciferase activity of the resulting moistened filter paper sample is measured.

FIG. 2 shows the properties of a CBD-BAF-bound paper sample dried and stored at room temperature.

(A) shows an example in which the CBD-BAF-Ym3-bound paper sample (filter paper), which had been dried and stored at room temperature, was reacted with a buffer and a luciferin aqueous solution.

(a), (b), and (c) respectively show a bright field image, merged image, and chemiluminescent image. In the bright field image (a), the round area in the center of the filter paper sample is an area where CBD-BAF-Ym3 was applied. In the merged image (b) and chemiluminescent image (c), a green emission was observed in the CBD-BAF-applied area.

(B) shows the results of luminescence intensity measured through a storage period (N=5). The horizontal axis indicates the period (in weeks) of a filter paper sample stored under room temperature drying conditions. The vertical axis indicates the luminescence intensity (×10⁸ RLU) measured. The dried filter paper was stored at room temperature of 27° C.

FIG. 3 shows the properties of an hCBD-eBAF-Ym3-bound paper sample dried and stored at room temperature for a long period of time.

(A) is a diagram showing the structure of hCBD-eBAF-Ym3. The structure includes, sequentially from the N terminal, hCBD, which is a cellulose- and/or chitin-binding domain, and eBAF-Ym3, which is a luminescent domain.

(B) shows the results of luminescence intensity measured through a storage period (N=5). The horizontal axis indicates the period (in weeks) of a filter paper sample stored under room temperature drying conditions. The vertical axis indicates the luminescence intensity (RLU) measured. The dried filter paper was stored at room temperature of 22 to 27° C. Note that a storage period of 52 weeks indicates one year.

FIG. 4 shows the properties of an hCBD-eBAF-R₃-bound paper sample having a protease recognition sequence dried and stored at room temperature for a long period of time.

(A) is a diagram showing the structure of hCBD-eBAF-R₃. The structure includes, sequentially from the N terminal, hCBD, which is a cellulose- and/or chitin-binding domain, a linker in which an HRV-3C cleavage sequence, which is a protease recognition sequence, is introduced, and eBAF-R₃, which is a luminescent domain.

(B) shows the results of luminescence intensity measured through a storage period (N=3). The horizontal axis indicates the period (in weeks) of a filter paper sample stored under room temperature drying conditions. The vertical axis indicates the luminescence intensity (RLU) measured. The dried filter paper was stored at room temperature of 22 to 26° C. Note that a storage period of 52 weeks indicates one year.

FIG. 5 shows the properties of an hCBD-eBAF-R₄-bound paper sample having a protease recognition sequence dried and stored at room temperature for a long period of time.

(A) is a diagram showing the structure of hCBD-eBAF-R₄. The structure includes, sequentially from the N terminal, hCBD, which is a cellulose- and/or chitin-binding domain, a linker in which an HRV-3C cleavage sequence, which is a protease recognition sequence, is introduced, and eBAF-R₄, which is a luminescent domain.

(B) shows the results of luminescence intensity measured through a storage period (N=3). The horizontal axis indicates the period (in weeks) of a filter paper sample stored under room temperature drying conditions. The vertical axis indicates the luminescence intensity (RLU) measured. The dried filter paper was stored at room temperature of 26° C. Note that a storage period of 52 weeks indicates one year.

FIG. 6 shows the procedure for an example of a protease activity detection model.

(A) (a-1) A chimeric protein aqueous solution was added dropwise to a filter paper sample to bind a chimeric protein to the filter paper. The paper was dried to produce a chimeric protein-bound filter paper sample (i).

(a-2) After adding a reaction buffer (ii) thereto to fully moisten the chimeric protein-bound filter paper sample (i), the buffer was removed, and a protease-containing buffer solution (iii) was further added.

(a-3) The resulting sample was allowed to stand at 4° C. for 64 hours to perform a protease reaction.

(B) (b-1) schematically shows the state before reaction. In the buffer, the chimeric protein-bound filter paper (paper sample) sank to the bottom of a micro centrifugal tube. The buffer included protease (p).

(b-2) The reaction proceeded by stationary incubation.

(b-3) schematically shows the state after reaction. The BAF portions cut by protease were separated into a water layer.

(b-4) The supernatant of the sample after reaction was collected by separation or separated to measure luminescence (or fluorescence).

FIG. 7 shows the results of an example of a protease activity detection model.

(A) schematically shows the mechanism of the model.

(a-1) shows the state where a chimeric protein is bound to a filter paper. The chimeric protein includes, sequentially from the N terminal, hCBD, which is a cellulose- and/or chitin-binding domain, a linker in which a HRV-3C cleavage sequence, which is a protease recognition sequence, is introduced, and BAF, which is a luminescent domain. The HRV-3C cleavage sequence is LEVLFQ/GP (“/” means a cleavage site).

(a-2) The chimeric protein was cut by an HRV-3C protease effect, and the BAF portion was separated into a water layer.

(B) shows the chemiluminescent measurement results of the collected supernatant (water phase). The vertical axis indicates the relative light intensity (RLU) measured. The water layer of the sample (+) to which a cleavage enzyme (HRV-3C protease) was added exhibited luminescence that is 3,000 times higher than the water layer of the sample (−) to which no cleavage enzyme was added.

(C) shows the results of SDS-PAGE electrophoresis. Samples are, from the left, the water layer of the sample (−) to which no cleavage enzyme (protease) was added, the water layer of the sample (+) to which a cleavage enzyme was added, and an hCBD-HRV3Cs-eBAF-Ym3 purified preparation (hCBD-BAF, control). The detected bands respectively show (i) hCBD-BAF, (ii) a BAF portion cut and separated from the filter paper into the water layer, and (iii) an HRV-3C enzyme.

(D) shows the GFP fluorescence of BAF in the water layer. The left side shows the water layer of the sample to which no cleavage enzyme (protease) was added and the right side shows the water layer of the sample to which a cleavage enzyme was added.

FIG. 8 shows an example of a chitin-binding domain sequence.

(A) shows an example of an amino acid sequence of chitin-binding domain 2 (chBD2) derived from Pyrococcus furiosus, and a base sequence encoding the amino acid sequence.

(B) shows an example of a chBD2 (TN) amino acid sequence in which Glu (E279) and Asp (D281) in the chBD2 amino acid sequence were respectively replaced by Thr (T) and Asn (N), and a base sequence encoding the amino acid sequence.

FIG. 9 shows the properties of a chimeric protein-binding chitin material having a protease recognition sequence dried and stored at room temperature for a long period of time.

(A) Chimeric proteins of hCBD-eBAF-Ym3, hCBD-eBAF-R₃, and hCBD-eBAF-R₄ were individually applied to a chitin material derived from a crab shell. FIG. 9(A) shows areas to which each of the chimeric proteins was applied.

(B) (b-1) shows a bright field image of a luminescent protein-chitin hybrid material stored for three days, which was obtained under a fluorescent lamp.

(b-2) shows a fluorescence image of the hybrid material stored for three days, which was obtained by excitation light irradiation.

(b-3) shows a fluorescence image obtained after storage for 10 months.

FIG. 10 shows the properties of an hCBD-RLuc-bound paper sample having a protease recognition sequence dried and stored at room temperature for a long period of time.

(A) is a diagram showing the structure of hCBD-RLuc. The structure includes, sequentially from the N terminal, hCBD, which is a cellulose- and/or chitin-binding domain, a linker in which an HRV-3C cleavage sequence, which is a protease recognition sequence, is introduced, and RLuc, which is a luminescent domain.

(B) shows the results of luminescence intensity measured through a storage period (N=3). The horizontal axis indicates the storage period (in weeks) of a filter paper sample under room temperature drying conditions. The vertical axis indicates the luminescence intensity (RLU) measured. The dried filter paper was stored at room temperature of 26° C.

FIG. 11 shows a luminescence spectrum of eBAF-Ym3, and the chemical luminescent activity (green) of an hCBD-eBAF-Ym3-bound chitin material obtained when a luciferin (luminescent substrate) was added. A chitin material derived from a crab shell was used.

(A) shows a luminescence spectrum of eBAF-Ym3. The horizontal axis indicates the wavelength (nm). The vertical axis indicates the relative light intensity (relative intensity) measured.

(B) (b-1), (b-2), and (b-3) respectively show a bright field image, a chemiluminescent image, and an overlapping image (an explanatory figure) of an example of an hCBD-eBAF-Ym3-bound chitin hybrid material. In (b-3), the area to which hCBD-eBAF-Ym3 was applied is indicated by an arrow.

FIG. 12 shows a luminescence spectrum of eBAF-R₃, and the chemical luminescent activity (orange) of an hCBD-eBAF-R₃-bound chitin material obtained when a luciferin (luminescent substrate) was added. A chitin material derived from a crab shell was used. (A) shows a luminescence spectrum of eBAF-R₃. The horizontal axis indicates the wavelength (nm). The vertical axis indicates the relative light intensity (relative intensity) measured.

(B) (b-1), (b-2), and (b-3) respectively show a bright field image, a chemiluminescent image, and an overlapping image of an example of an hCBD-eBAF-R₃-bound chitin hybrid material.

FIG. 13 shows a luminescence spectrum of eBAF-R₄, and the chemical luminescent activity (white) of an hCBD-eBAF-R₄-bound chitin material obtained when a luciferin (luminescent substrate) was added. A chitin material derived from a crab shell was used.

(A) shows a luminescence spectrum of eBAF-R₄. The horizontal axis indicates the wavelength (nm). The vertical axis indicates the relative light intensity (relative intensity) measured.

(B) (b-1), (b-2), and (b-3) respectively show a bright field image, a chemiluminescent image, and an overlapping image of an example of an hCBD-eBAF-R₄-bound chitin hybrid material.

FIG. 14 shows the luminescence of an hCBD-eBAF-Ym3-bound chitin hybrid material. A cicada exuvia was used as a chitin material.

(A) shows a comparison between the hCBD-eBAF-Ym3-bound material and the control.

(a-1) shows a bright field image and (a-2) shows a chemiluminescent image. The left side shows a container holding the hCBD-eBAF-Ym3-bound material, and the right side shows a container holding a buffer alone (control).

(B) shows an entire image of a cicada exuvia to which hCBD-eBAF-Ym3 was bound. (b-1) and (b-2) respectively show a bright field image and a chemiluminescent image.

### DESCRIPTION OF EMBODIMENTS

The chimeric protein of the present invention comprises a domain that binds to cellulose and/or chitin (a cellulose/chitin-binding domain) and a luminescent domain.

### (1) Cellulose/Chitin-Binding Domain

The cellulose/chitin-binding domain may be any domain capable of binding to cellulose (a cellulose-binding domain), any domain capable of binding to chitin (a chitin-binding domain), and any domain capable of binding to both cellulose and chitin.

Examples of cellulose-binding domains include those found in cellulase. A number of cellulose-binding domains derived from various living organisms, such as microorganisms, plants, and animals, are known. These known cellulose-binding domains can be widely used.

Examples of chitin-binding domains include those found in chitinase. A number of chitin-binding domains derived from various living organisms, such as microorganisms, plants, and animals, are known. These known chitin-binding domains can be widely used.

Specific examples of chitin-binding domains include those found in chitinase from heat-resistant bacteria. Examples of heat-resistant bacteria include those belonging to the genus Thermococcus or Pyrococcus. Specific examples of heat-resistant bacteria include Pyrococcus furiosus, Thermococcus litoralis, Pyrococcus sp.KOD1, and Thermotoga maritima. The amino acid sequence of SEQ ID No. 10 is chitin-binding domain 2 (ChBD2) from Pyrococcus furiosus, which is one of the preferable chitin-binding domains. This region corresponds to the region from the 258th to the 352nd amino acids of chitinase from Pyrococcus furiosus.

Examples of cellulose/chitin-binding domains (i.e., domains capable of binding to both chitin and cellulose) include those derived from heat-resistant bacteria as described in JP2007-075046A. Specific examples include heat-resistant cellulose/chitin-binding domains obtained by introducing a mutation into the heat-resistant chitin-binding domains of such heat-resistant bacteria.

A specific example of a cellulose/chitin-binding domain is an amino acid sequence that obtained by replacing two acidic amino acids (E279 and D281) of the amino acid sequence (SEQ ID No. 10) of Pyrococcus furiosus-derived chitin-binding domain 2 (ChBD2) with other amino acids, wherein the amino acid sequence encodes a polypeptide having cellulose-binding activity. Examples of other amino acids to replace acidic amino acids include neutral amino acids with low hydrophobicity, typically Gln, Asn, Ala, Ser, Thr, Cys, and Met, preferably Gln, Asn, Ala, Ser, Thr, and Cys, and more preferably Gln, Asn, Ala, Ser, and Thr. Further, Glu(E279) is more preferably replaced by Thr(T), and Asp(D28) is more preferably replaced by Asn(N). A specific example is ChBD2(TN), which is a sequence obtained by replacing Glu(E279) and Asp(D281) of the ChBD2 amino acid sequence with Thr(T) and Asn(N), respectively (FIG. 8, SEQ ID No. 11).

### (2) Luminescent Domain

Examples of luminescent domains include a variety of luciferases, fluorescent proteins, and fused proteins thereof (e.g., BAF). Examples of luciferases include a variety of luciferases derived from Lampyridae (fireflies), Rhagophthalmus ohbai, Vargula hilgendorfii, Phrixothrix hirtus, Pyrearinus termitilluminans, dinoflagellate, Renilla and the like. Examples of fluorescent proteins include GFP, YFP, BFP, CFP, OFP, DsRED, and RFP.

Either a luciferase or a fluorescent protein may be singly used as a luminescent domain. A preferable luminescent domain is a protein in which a luciferase is bound to a fluorescent protein directly or via a spacer of a proper length, and energy transfer (BRET: bioluminescence resonance energy transfer) between the luciferase and the fluorescent protein occurs, i.e., a BAF protein (simply referred to as “BAF”). A process for producing DNA encoding BAF is described, for example, in PTL 2. Such a DNA is obtained by binding a luciferase gene to a fluorescent protein gene via a proper DNA sequence corresponding to the second linker. In this specification, a chimeric protein having BAF as a luminescent domain may be referred to as “CBD-BAF.”

### (3) Chimeric Protein

The chimeric protein of the present invention is obtained by incorporating a genetic construct or vector into a host cell (e.g., Escherichia coli) to thereby produce a transformant, and culturing the transformant, wherein the genetic construct or vector comprises a chimeric protein-encoding DNA formed by bonding DNA encoding a cellulose/chitin-binding domain to DNA encoding a luminescent domain directly or via a DNA sequence corresponding to the first linker.

### (4) Linker

The first linker is not particularly limited as long as it comprises one or more amino acids, and as long as it does not impair the function of the cellulose/chitin-binding domain and the luminescent domain. The number of amino acids constituting the first linker is at least 1, and may range from 2 to 100, for example, 4 to 80, preferably 5 to 60, more preferably about 6 to 40, still more preferably 7 to 30, and particularly more preferably about 8 to 16.

The second linker is not particularly limited as long as it comprises one or more amino acids, and as long as it does not interrupt energy transfer from a luciferase to a fluorescent protein. The number of amino acids constituting the second linker is typically 8 to 26, preferably 8 to 16, more preferably 10 to 14, and particularly more preferably 12. When the number of amino acids constituting the linker is 7 or less, or 27 or more, the energy transfer efficiency is significantly lowered.

A protease recognition sequence can be incorporated into the linker (the first linker or the second linker). Such makes it possible to detect whether a protease is present in a sample by using the chimeric protein of the present invention. Alternatively, an amino acid sequence which enables one or more substances in the sample bind to the linker to thereby change the luminescent activity can be incorporated into the into the linker. Such makes it possible to detect or quantify such a linker-binding substance in the sample. Such a protease recognition sequence and linker-binding substance are known, and can be suitably selected by a person skilled in the art. A specific example of a combination of a protease and a protease recognition sequence includes, but is not limited to, HRV-3C protease and an amino acid sequence LEVLFQ/GP (“/” means the cleavage site).

### (5) Others

In this specification, luciferases to be used may be wild-type luciferases. Luciferases with improved properties, such as stability and luminescence, may also be used.

In this specification, fluorescent proteins to be used may be wild-type fluorescent proteins. Fluorescent proteins with improved properties, such as stability and luminescence, may also be used.

When using, for example, Renilla luciferase as a luminescent domain, a wild-type Renilla luciferase (e.g., Rluc) may be used. Renilla luciferases with improved properties, such as stability and luminescence (e.g., Rluc8 and Rluc8/A123S/D162E/I163L) may also be used. As used herein, the term “luciferase” includes both wild-type luciferases and any luciferases that have modified luciferase properties. Likewise, as used herein, the term “fluorescent protein” includes both wild-type fluorescent proteins and any fluorescent proteins that have modified properties of a fluorescent protein.

Examples of fluorescent protein include green fluorescent protein (GFP), yellow fluorescent protein (YFP), blue fluorescent protein (BFP), cyan fluorescent protein (CFP), orange fluorescent protein (OFP), DsRED, and red fluorescent protein (RFP). GFP includes wild-type green fluorescent protein (e.g., AvGFP and AcGFP) derived from jellyfish of the genus Aequorea (e.g., Aequorea victoria, and Aequorea coerulescens) and various GFP derivatives, such as EGFP. YFP also includes a wide variety of variants comprising one or more amino acid substitutions, such as EYFP, Topaz, Venus, and Citrine. DsRED includes a wide variety of wild-type fluorescent proteins derived from coral of the genus Discosoma, their variants having their amino acid sequences modified, e.g., by substitution, addition, deletion or insertion, and monomeric forms obtained by modifying polymeric wild-type DsRED (e.g., mCherry). DsRED of monomeric form is preferable. RFP includes a wide variety of wild-type red fluorescent proteins derived from, for example, actiniae (e.g., Entacmaea quadricolor; note, however, that DsRED that emits red light is not included) and their variants having their amino acid sequence modified (e.g., TurboRFP). Likewise, other fluorescent proteins also include a wide variety of wild-type fluorescent proteins and their variants having their amino acid sequences modified, e.g., by substitution, addition, deletion or insertion.

The use of a fluorescent protein that changes RLU (relative light unit, relative intensity) or fluorescence wavelength depending on pH, such as YFP, makes it possible to measure the pH of the environment where the fluorescent protein is present. Thus, the chimeric protein of the present invention can be used as a pH indicator. Further, the use of proteins that do not significantly change RLU or wavelength depending on pH, such as GFP, makes it possible to quantify, without being affected by the pH, the chimeric protein of the present invention or substances (e.g., proteins) labeled by such a chimeric protein.

The DNA of the present invention encodes the chimeric protein of the present invention.

The chimeric protein of the present invention is obtained by incorporating the gene of the present invention, which will be described below, into an expression vector, and allowing gene expression to occur in an appropriate host cell. Examples of the host cell include mammal and other animal cells, plant cells, eukaryotic cells (e.g., yeast) and prokaryotic cells (e.g., Escherichia coli, Bacillus subtilis, algae, and Eumycetes). Any of these cells can be used. Preferable host cells include Escherichia coli and the like.

A feature of the chimeric protein of the present invention is that the chimeric protein, when attached or bound to a material, such as a sheet, a film, a granule, or a bead, which is formed of cellulose or chitin, and dried, maintains its luminescence activity for a long period of time. Thus, the chimeric protein of the present invention is useful as a luminescent material. Further, due to its luminescent activity not being lowered in storage for a long period of time, the chimeric protein of the present invention is useful as a standard substance as well.

In this specification, cellulose to be used includes wild-type cellulose and regenerated cellulose. Examples of wild-type cellulose include refined pulp obtained from needle-leaved trees or broad-leaved trees, cellulose obtained from cotton linter or cotton lint, cellulose obtained from sea grass, such as Valoniaceae and Cladophorale, cellulose obtained from ascidian, and cellulose produced by bacteria. Examples of regenerated cellulose include those obtained by dissolving natural cellulose fibers and regenerating them in a fibrous form with the chemical structure of cellulose maintained.

Chitin can be obtained from, for example, crab shells. According to a preferred embodiment of the invention, chitin is obtained by the following procedure: a crab shell is washed with water, and treated with an acid, such as hydrochloric acid to remove inorganic matter (e.g., calcium); subsequently, organic matter (e.g., protein) is removed by a caustic soda treatment, and lipid is further removed by an alcohol treatment, thereby obtaining chitin as an insoluble residue for use. The crab shell material pulverized into granules may also be used.

Further, cicada exuviae can be used as chitin. Because chitin is exposed on the inner surface of exuviae, cicada exuviae can be used without the need for treatment.

### EXAMPLES

The present invention is described in detail below using Examples. Needless to say, however, the present invention is not limited to these Examples. In the Examples, it is understood that “CBD-BAF” and “hCBD-BAF” are included in “chimeric proteins.”

### Example 1

### Production of Plasmid

(1) pCII-CBD-eBAF-Ym3 and pCII-CBD(TN)-eBAF-Ym3

In order to produce CBD-eBAF-Y expression vectors, the gene coding for CBD(wt) or CBD(TN) was amplified by PCR. The primers used for PCR were as follows:

chBD2-F-NdeI,
5′-GGAATTCCATATGACTACCCCTGTCCCAGTCTC-3′;
and
chBD2-R-NdeI,
5′-CGATATCCATATGAATTACTTGTCCGTTTATTTCTAG-3′.

The PCR fragments were digested with NdeI, and inserted into the NdeI site of pCII-eBAF-Ym3, thereby building pCII-CBD-eBAF-Ym3 and pCII-CBD(TN)-eBAF-Ym3.

eBAF-Ym3 is described in PTL 1, and is a BAF protein in which mutations of A123S, D162E, and I163L are introduced into the RLuc8 portion of eBAF-Y. The genes coding for CBD and CBD(TN) are sequences derived from the genome of a hyperthermophilic bacterium.

(2) pCII-hCBD-eBAF-Y

For efficient protein expression in Escherichia coli, the CBD gene (only CBD(TN)) was artificially synthesized for the purpose of codon optimization in Escherichia coli. The artificial synthetic CBD(TN) gene (hereinafter referred to as “hCBD” for distinction; however, the amino acid sequence thereof was the same as that of CBD(TN)) was used to replace the CBD(TN) portion of the above pCII-CBD(TN)-eBAF-Ym3, thereby building pCII-hCBD-eBAF-Ym3. Further, at this time, in preparation for recombination at a later stage, an Asp718-BamHI-NdeI site was added to the 3′ end of the hCBD sequence when the artificial gene was designed. As a result, the nucleotide sequence of the junction region was 5′-GGTACCGGGGGATCCCATATG-3′, and hCBD and eBAF-Ym3 were connected in-frame via the amino acid sequence G-T-G-G-S-H (ATG of the NdeI site corresponded to the initiation Met of eBAF-Ym3).

(3) pCII-hCBD-HRV3Cs-eBAF-Ym3

A synthetic double-stranded DNA comprising AspHRV3CsBam-Sens: 5′-GTACCGGTGGTTCCGCGGGTCTGGAAGTTCTGTTCCAGGGGCCCTCCGCGGGTtccggtg-3′ and AspHRV3CsBam-Anti: GATCCACCGGAACCCGCGGAGGGCCCCTGGAACAGAACTTCCAGACCCGCGGAACCACCG, was inserted into the Asp718-BamHI site of pCII-hCBD-eBAF-Ym3, and an HRV-3C protease cleavage sequence was inserted. The amino acid sequence corresponding to the region from the Asp718 site to the BamHI site was G-T-G-G-S-A-G-L-E-V-L-F-Q-G-P-S-A-G-S-G-G-S, and LEVLFQ/GP in the center was the protease cleavage sequence (/: cleavage site).

(4) pCII-hCBD-HRV3Cs-eBAF-Ym3ΔNdeI

In Escherichia coli expression vectors for various BAFs, typified by pCII-eBAF-Y, more than 400 types of various BAFs, which had been developed by the year 2008, are all cloned with the NdeI-XbaI site. Comparatively, in pCII-hCBD-HRV3Cs-eBAF-Ym3, the hCBD portion is inserted at the NdeI site, and the NdeI site on the 5′ end of the hCBD portion interrupts the production of new BAF substitution products. Accordingly, the NdeI site was destroyed by single-nucleotide substitution mutagenesis using hCBDdelNdeIoligo-Sens: 5′-TCATCATCATCATCAcATGACCACTCCGGTG-3′ and hCBDdelNdeIoligo-Anti: 5′-CACCGGAGTGGTCATgTGATGATGATGATGA-3′, and pCII-hCBD-HRV3Cs-eBAF-Ym3ΔNdeI was thereby produced. The QuikChange system (Stratagene) was used to perform the single-nucleotide mutagenesis.

FIG. 3 shows the test results of the luminescent activity of the chimeric protein expressed using pCII-hCBD-HRV3Cs-eBAF-Ym3ΔNdeI after it was adsorbed/bound to filter paper, and then dried.

(5) pCII-hCBD-HRV3Cs-eBAF-R₃ and pCII-hCBD-HRV3Cs-eBAF-R₄

pCII-hCBD-HRV3Cs-eBAF-Ym3ΔNdeI was digested with NdeI and XbaI to remove the eBAF-Ym3 portion, and BAF-R₃ or BAF-R₄ was inserted instead. Thus, pCII-hCBD-HRV3Cs-eBAF-R₃ and pCII-hCBD-HRV3Cs-eBAF-R₄ were produced. BAF-R₃ and BAF-R₄ contained TurboRFP and mCherry, respectively, as red fluorescent proteins. BAF-R₃ and BAF-R₄ were produced according to the method described in PTL 1.

FIGS. 4 and 5 show the test results of the luminescent activity of to the chimeric proteins expressed using pCII-hCBD-HRV3Cs-eBAF-R₃ and pCII-hCBD-HRV3Cs-eBAF-R₄ after they were each adsorbed/bound filter paper, and then dried.

(6) pCII-hCBD-RLuc

The eBAF-Ym3 portion was removed from pCII-hCBD-HRV3Cs-eBAF-Ym3ΔNdeI and RLuc (Renilla luciferase) was inserted instead, in the same manner as in (5) above. FIG. 10 shows the test results of the luminescent activity of the chimeric protein expressed using the obtained pCII-hCBD-HRV3Cs-RLuc after it was adsorbed/bound to filter paper, dried, and stored at room temperature.

### Preparation of Recombinant Protein

Each chimeric protein was expressed in an Escherichia coli BL21 strain by a low-temperature shock inducible promoter system (TAKARA) using a recombinant protein as a His-tagged fused protein. The recombinant protein was purified by a Ni-NTA affinity column.

### Production of Various Chimeric Protein-Bound Filter Papers

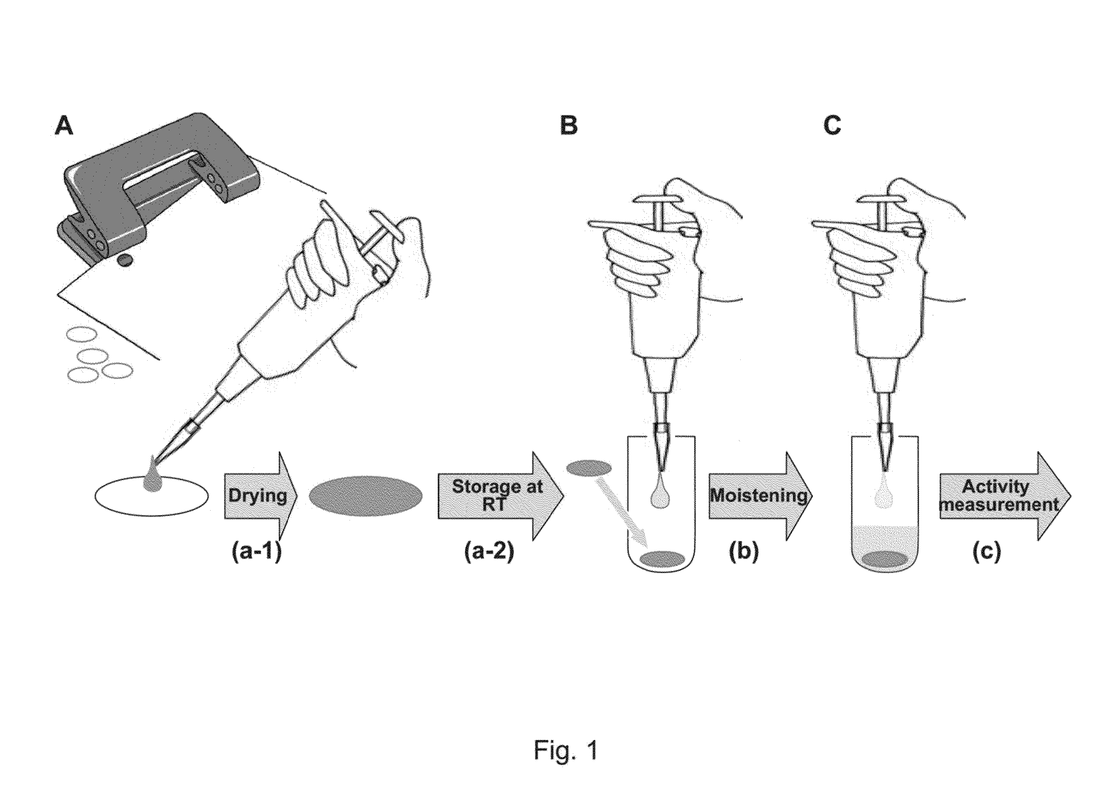

A round filter paper (ADVANTEC) sample (diameter: 6 mm) produced by a hole puncher was placed on a Parafilm sheet. A high-concentration aqueous solution of each chimeric protein, which had been His-tagged and purified, was added dropwise (several microliters per drop) to the filter paper sample and then dried. This process was repeated. After binding a sufficient amount of chimeric protein, the filter paper sample was washed with a large amount of purified water to remove unbound CBD-BAF. The washed filter paper sample was air-dried on a Parafilm sheet. Various chimeric protein-bound filter papers were produced in this manner. FIG. 1 schematically shows the outline of the method. The chimeric proteins used were CBD-eBAF-Ym3, hCBD-HRV3Cs-eBAF-Ym3ΔNdeI (hereinafter also referred to as “hCBD-eBAF-Ym3”), hCBD-HRV3Cs-eBAF-R₃ (hereinafter also referred to as “hCBD-eBAF-R₃”), hCBD-HRV3Cs-eBAF-R₄ (hereinafter also referred to as “hCBD-eBAF-R₄”), and hCBD-HRV3Cs-RLuc (hereinafter also referred to as “hCBD-RLuc”).

### Observation of Luminescence Image of Chimeric Protein-Bound Filter Paper

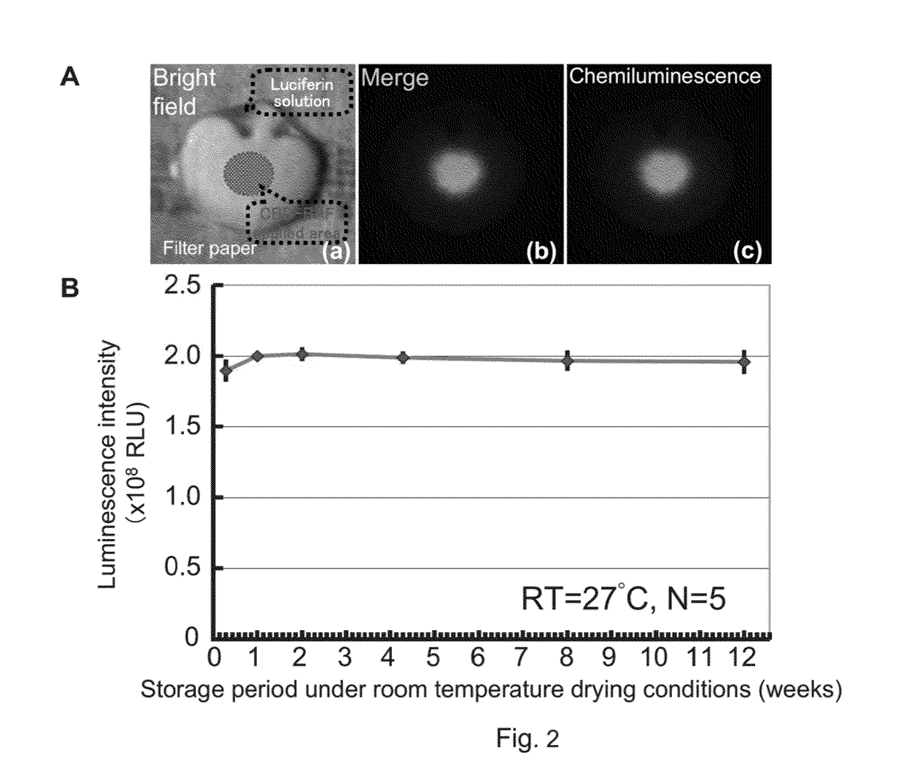

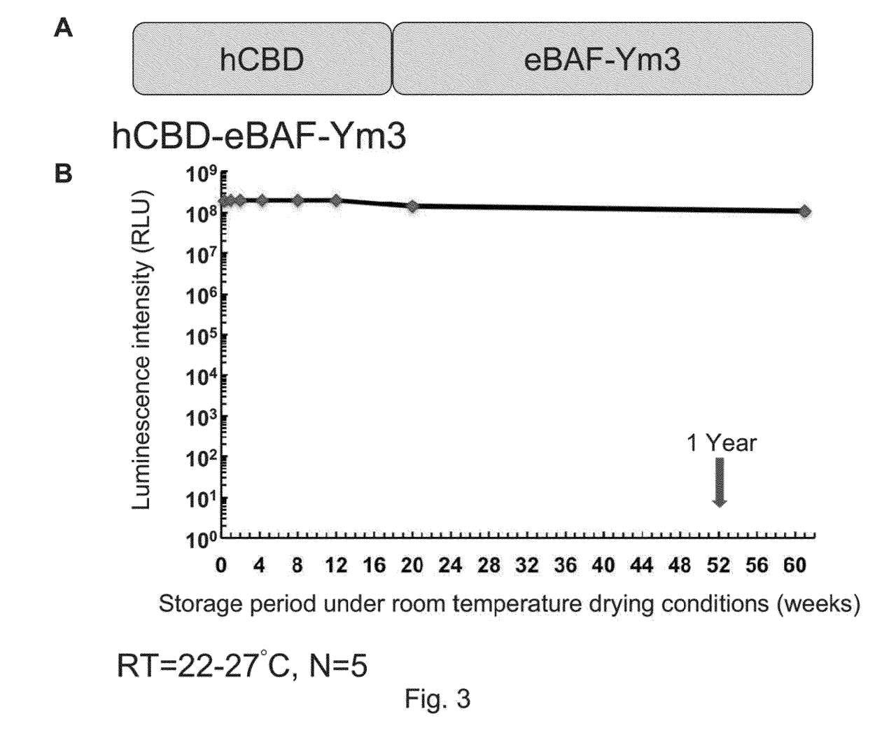

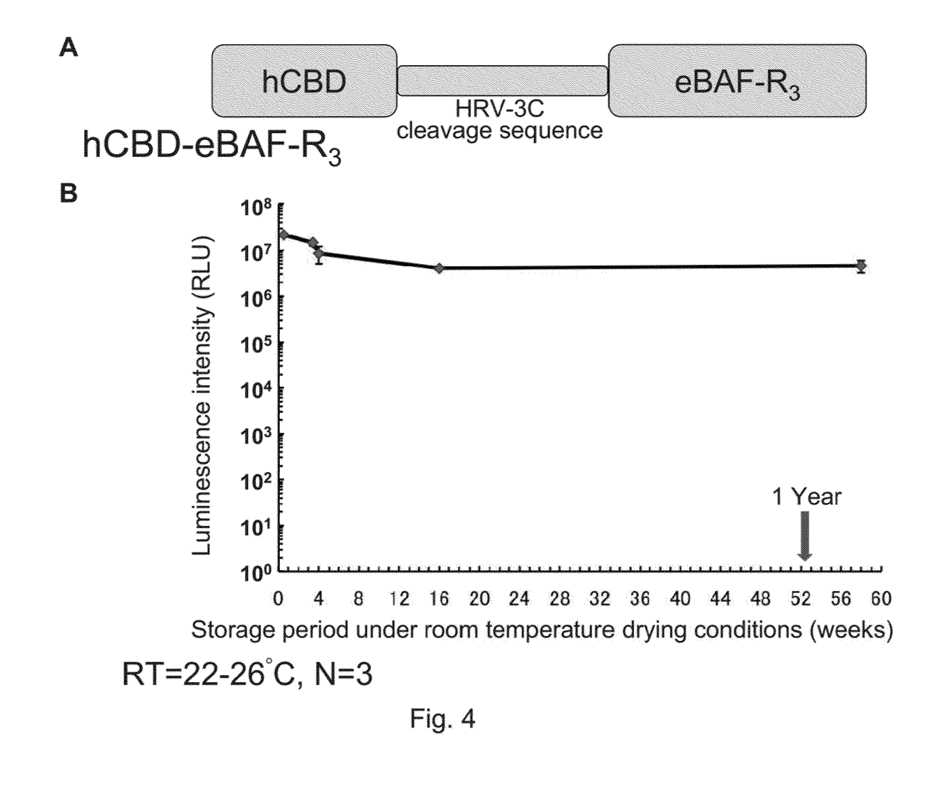

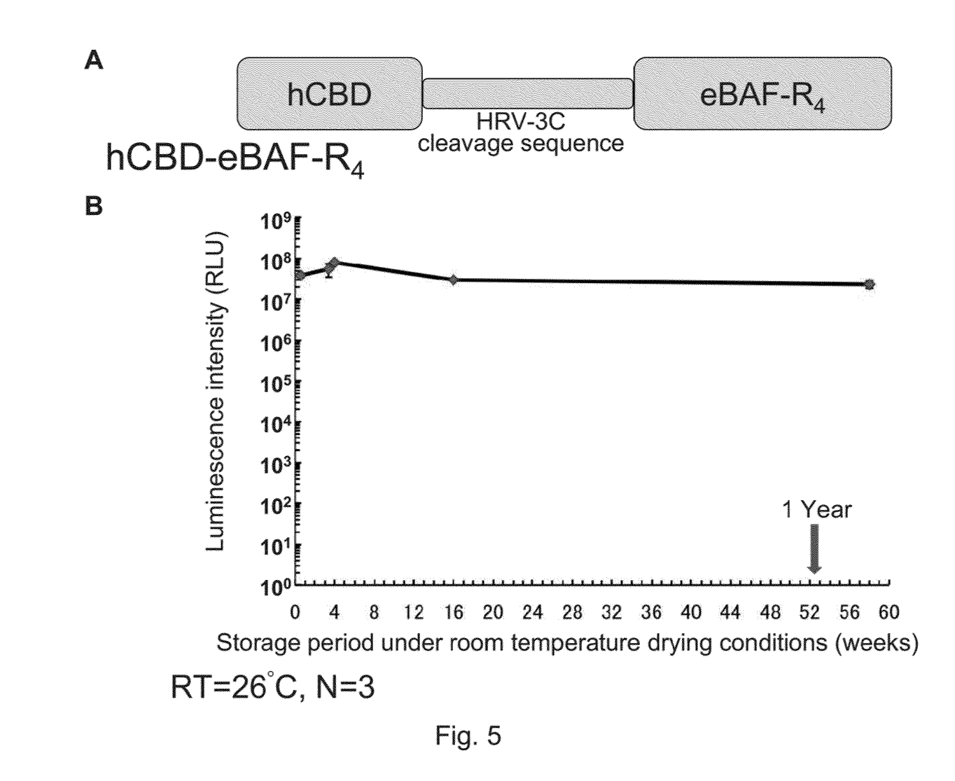

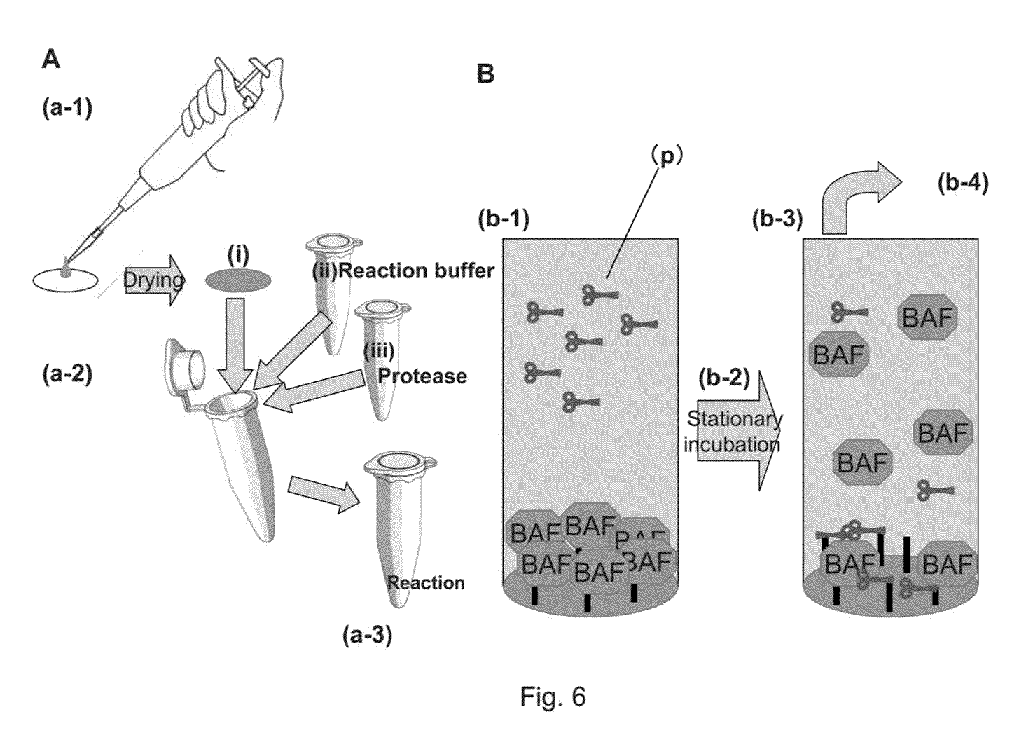

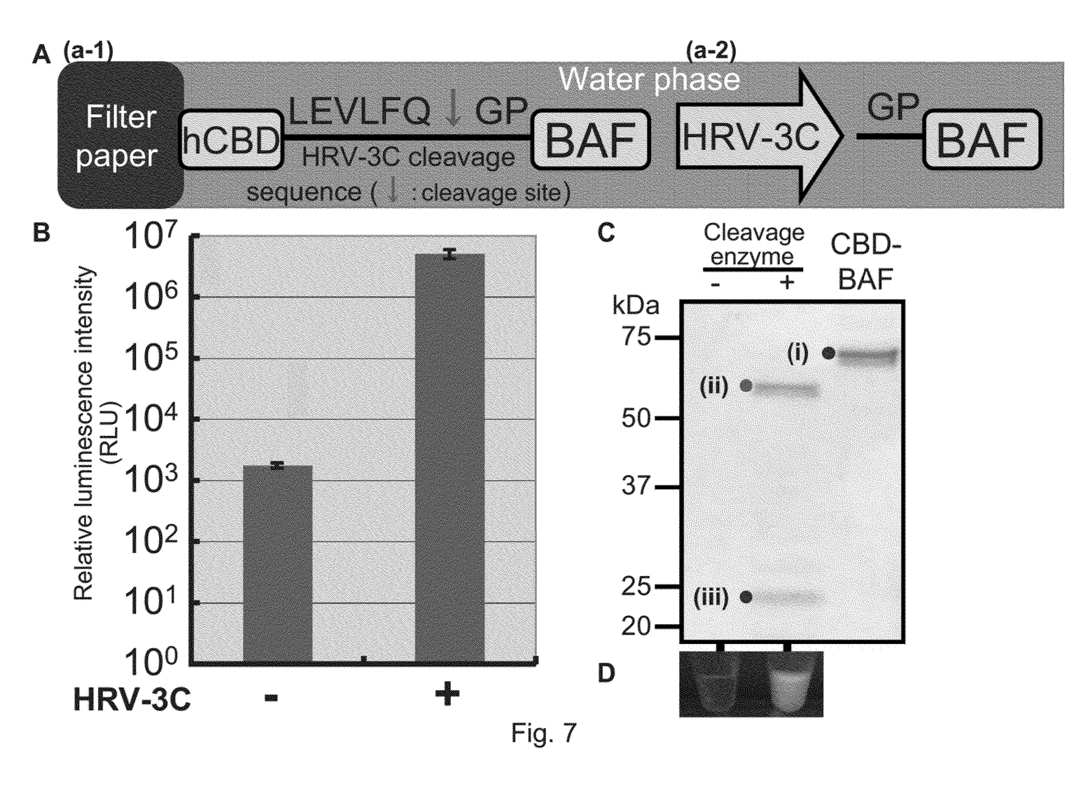

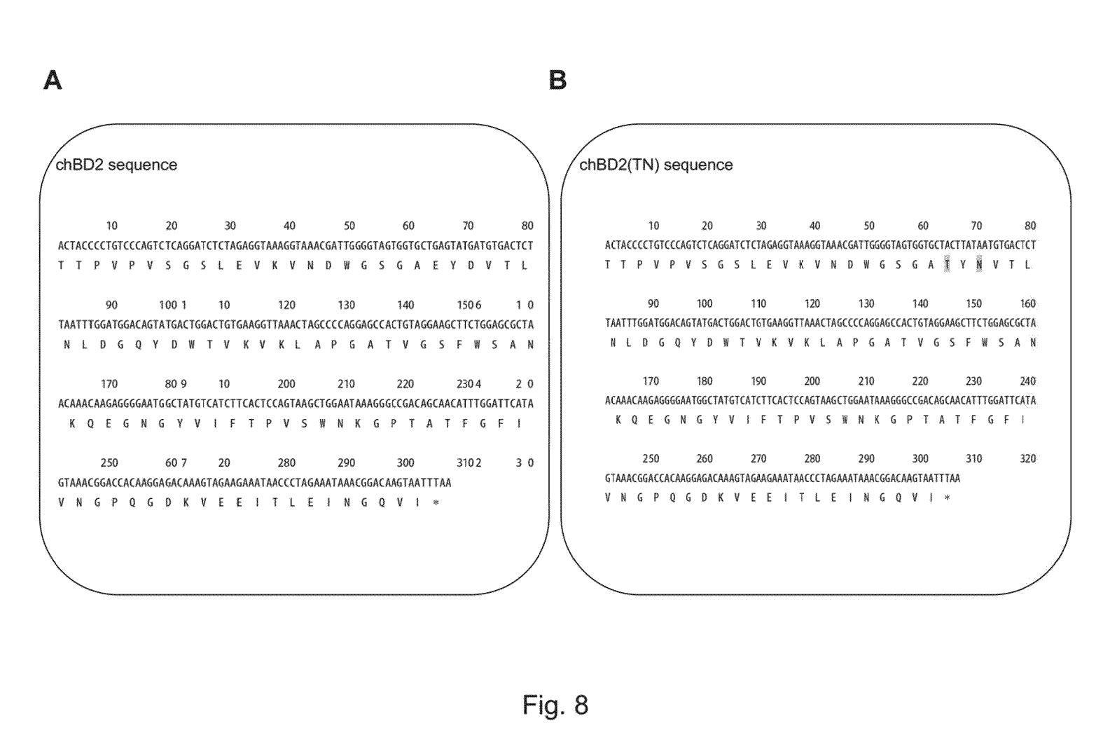

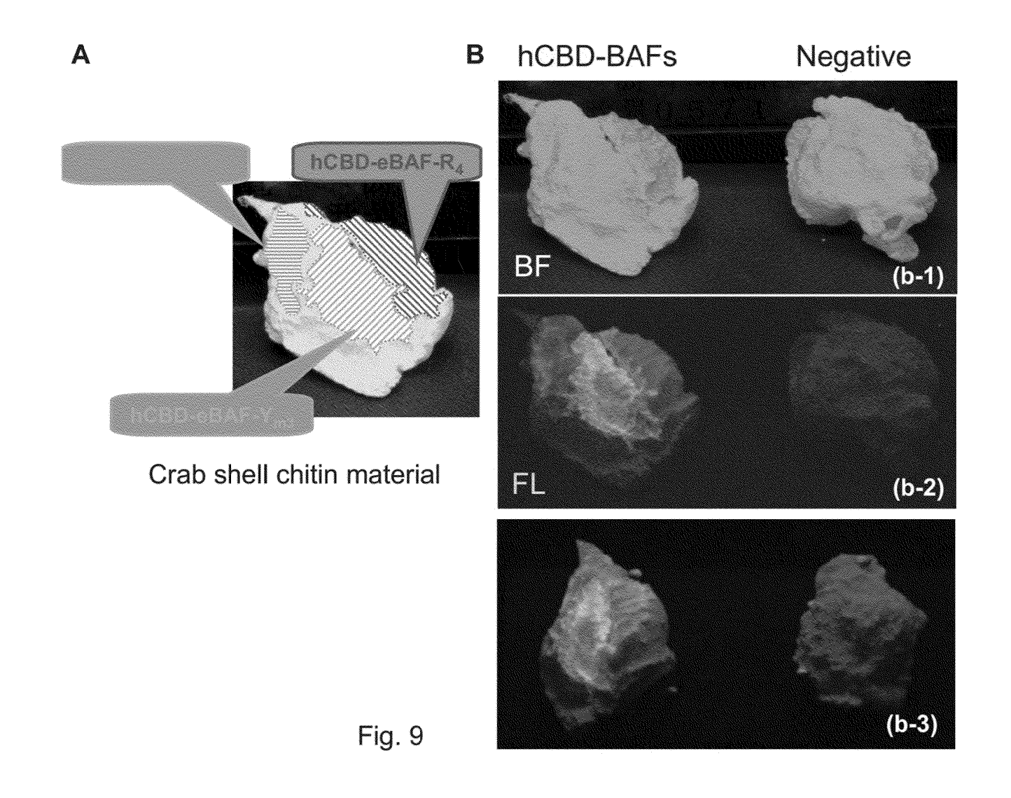

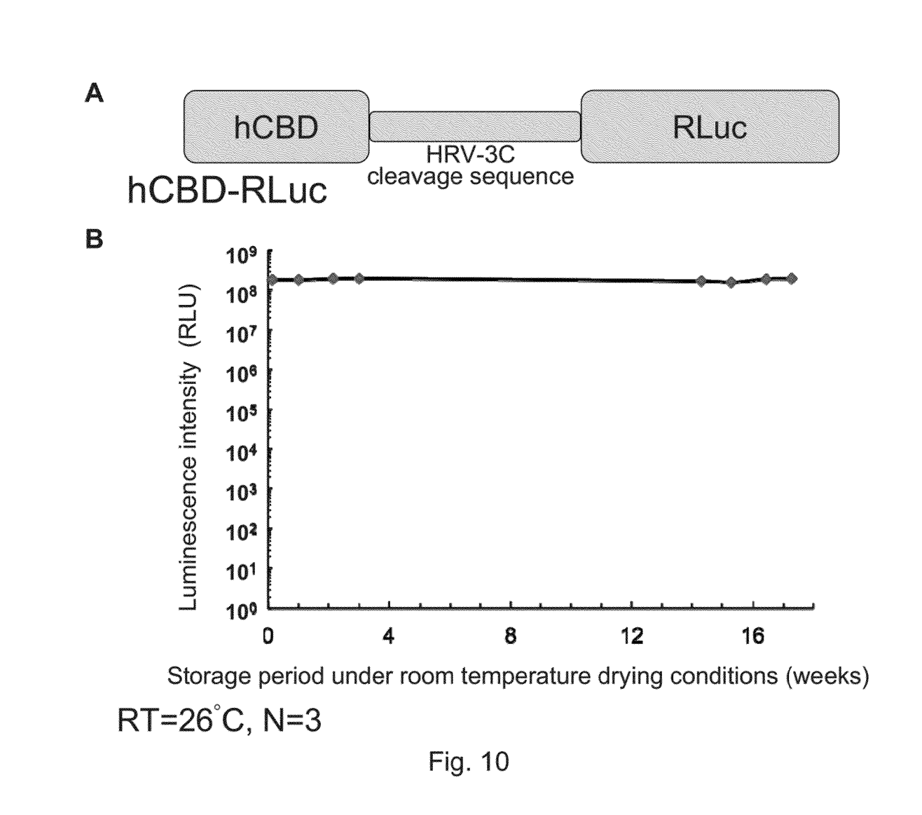

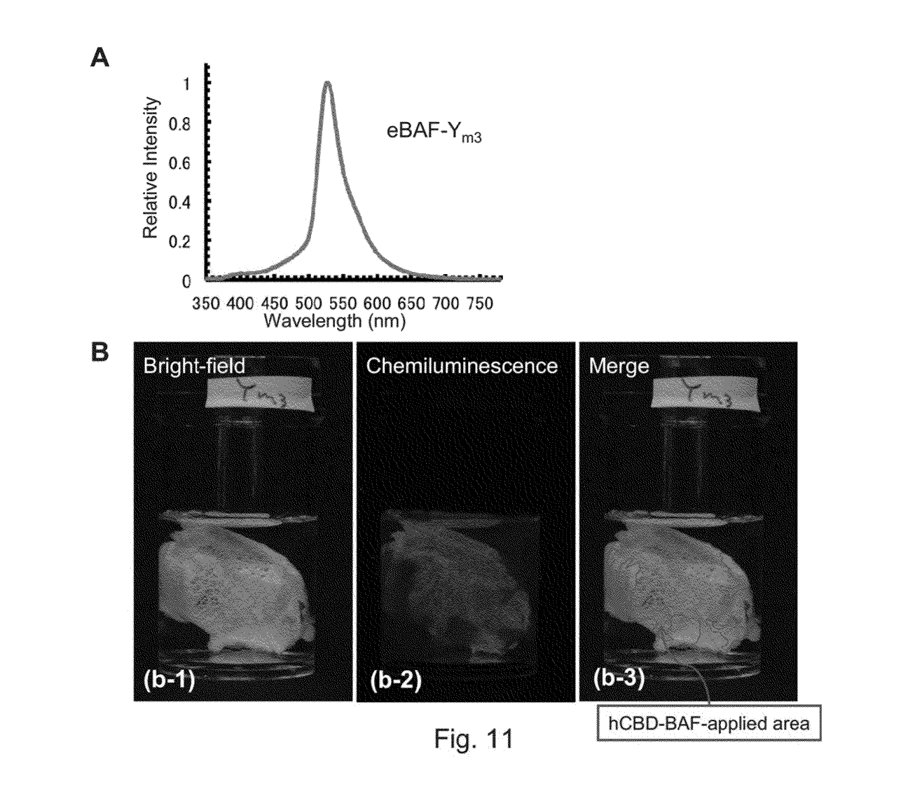

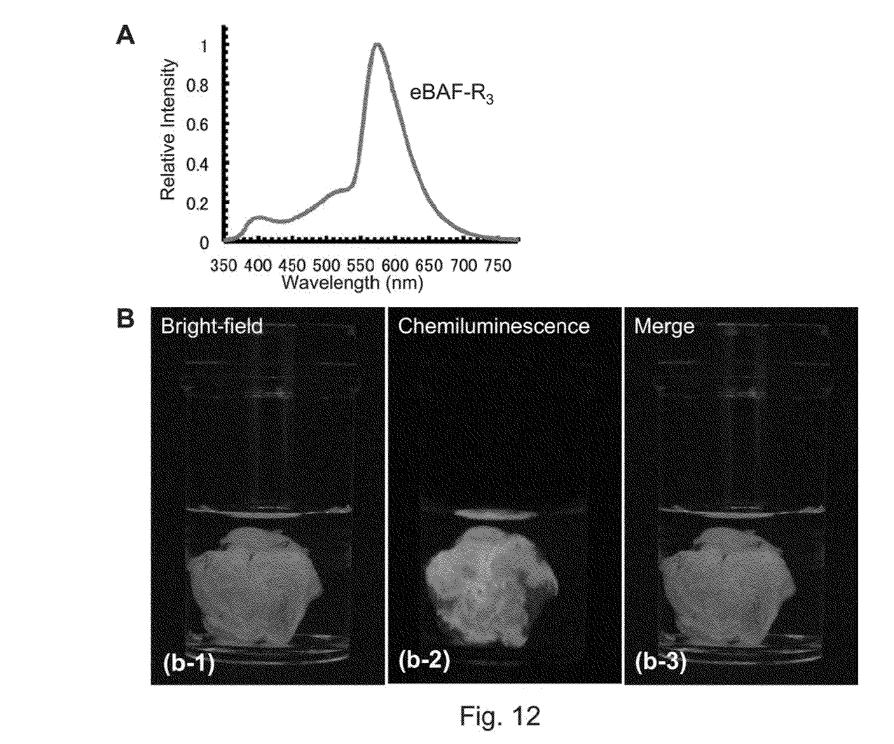

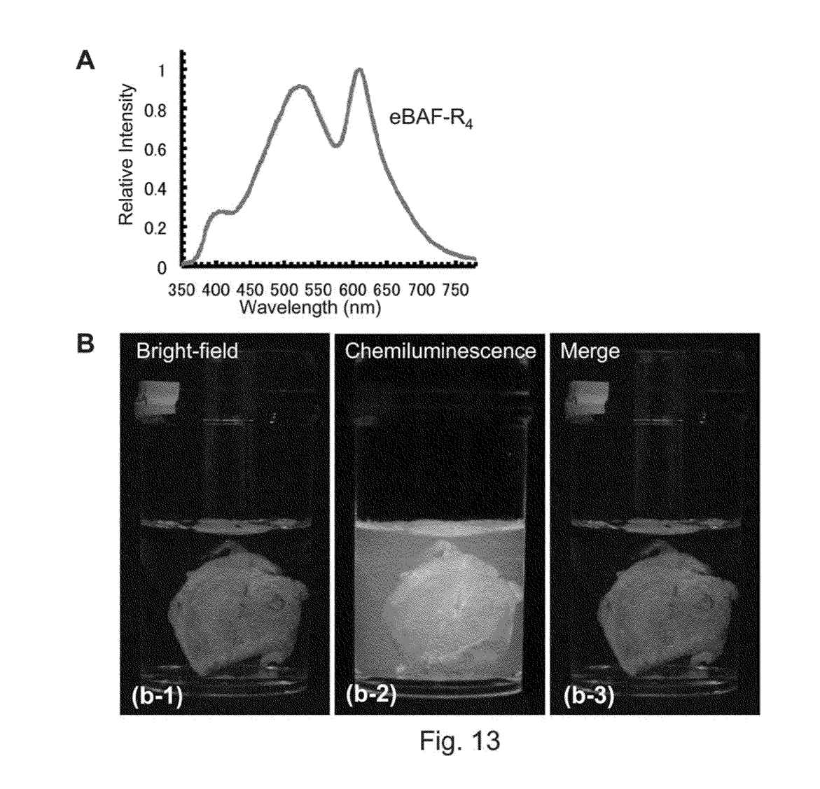

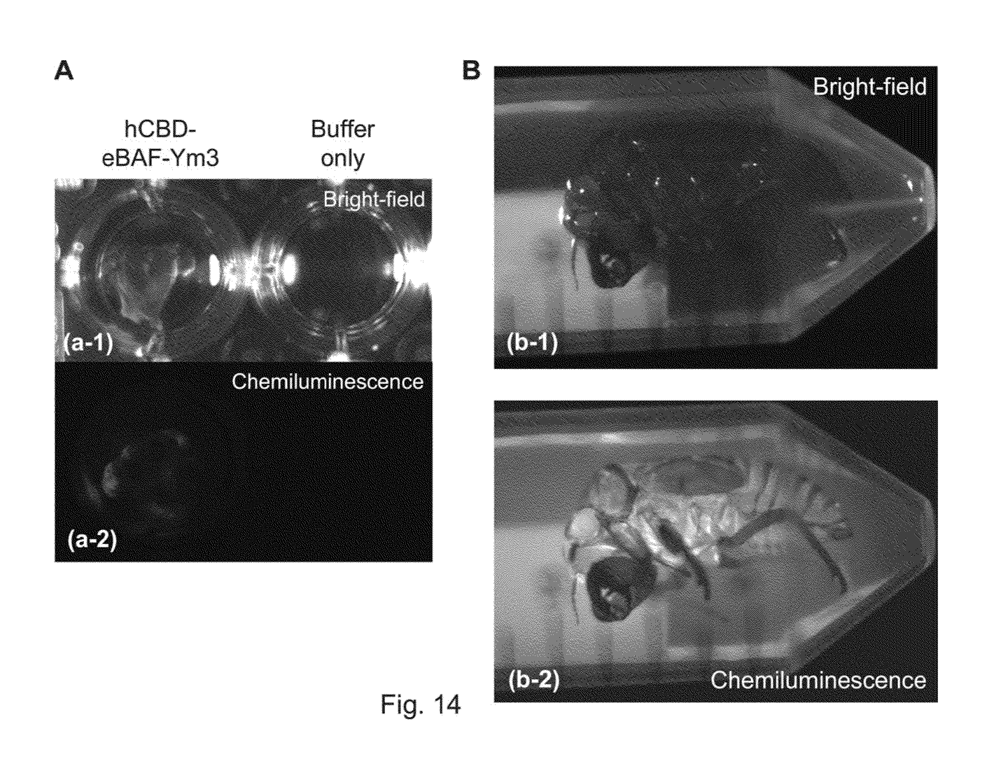

A filter paper sample was cut out by an apple-shaped hole puncher, and CBD-eBAF-Ym3 was bound to the center of the filter paper sample. After washing the filter paper sample, a luciferin solution was added thereto, and yellowish-green luminescence was visually observed. After it was confirmed that no diffusion from the part coated with the chimeric protein was observed, a luminescence image was acquired by a LAS-4000 with an exposure time of 4 seconds at High Resolution mode (lowest sensitivity). FIG. 2A shows the results.

Only in this Example, CBD-eBAF-Ym3 having CBD (chBD2(TN) type; the gene (base) sequence except for the mutated portion was derived from a natural hyperthermophilic bacterium) was used as the cellulose/chitin-binding domain. In all of the other Examples, an artificial synthetic gene (also referred to as “hCBD”) coding for the amino acid sequence of chBD2(TN) and optimized for codon usage in Escherichia coli was used.

Measurement of Luminescence Intensity after Dry Storage of Chimeric Protein-Bound Filter Paper at Room Temperature

The CBD-eBAF-Ym3 protein-binding filter paper was placed in a plastic petri dish, and the petri dish was covered with a lid and then stored at room temperature (26° C. to 27° C.) in a dark place. Immediately before measurement, the dry filter paper sample was placed in a luminometer measuring tube (Nunc), and 200 μl of luminescent reaction buffer (60 mM NaCl, 50 mM Tris-HCl, pH 8.0) was added to sufficiently moisten the sample. A 1 μM luciferin solution (200 μl) was added to the tube, and the luminescence was measured. The luminescence intensity was measured by integration for 10 seconds using a Luminescencer-PSN (Atto). FIG. 2B shows the results.

The luminescence intensity of hCBD-HRV3Cs-eBAF-Ym3ΔNdeI, hCBD-eBAF-R₃, hCBD-HRV3Cs-eBAF-R₄, and hCBD-HRV3Cs-RLuc after storage was similarly measured. FIGS. 3 to 5 and 10 show the results.

### Implementation of Protease Activity Detection System

FIGS. 6 and 7A schematically show the outline of the system.

The expression of hCBD-HRV3Cs-eBAF-Ym3 was induced using pCII-hCBD-HRV3Cs-eBAF-Ym3-containing Escherichia coli, followed by purification. The obtained hCBD-HRV3Cs-eBAF-Ym3 was used to prepare a dry filter paper sample to which this protein was bound.

The filter paper sample was placed in a 2.0-ml micro centrifugal tube, and 100 μl of 1×HRV-3C buffer (150 mM NaCl, 50 mM Tris-HCl, pH 7.5) was added to sufficiently moisten the sample. Then, the buffer was completely removed. To the tube containing the buffer-moistened filter paper sample, 120 μl of 1×HRV-3C buffer solution or 1×HRV-3C buffer solution containing 4U of HRV-3C protease (Novagen) was newly added, and the mixture was allowed to stand at 4° C. for 64 hours. After the reaction, the micro centrifugal tube was centrifuged, and 40 μl of supernatant was taken in another tube. The collected supernatant was supplied to SDS-PAGE each in an amount of 4 μl, and separated by electrophoresis, followed by CBB dyeing. In addition, an unreacted hCBD-HRV3Cs-eBAF-Ym3 purified preparation was used as a control. FIG. 7C shows the results.

Further, 2 μl of the collected supernatant was diluted with 200 μl of luminescent reaction buffer (60 mM NaCl, 50 mM Tris-HCl, pH 8.0), and an equivalent amount of 0.5 μM luciferin solution was added. The luminescence was measured three times. The luminescence intensity was measured by integration for 10 seconds using a Luminescencer-PSN (Atto). FIG. 7B shows the results.

Moreover, the fluorescence of the collected supernatant was observed using a blue LED transilluminator and an orange acrylic plate. An image was obtained by photography using a digital camera (Nikon D-70). FIG. 7D shows the results.

Production of Chitin Material to which hCBD-BAF Proteins Bind

A crab shell was sequentially treated with hydrochloric acid (calcium removal), NaOH (protein removal), and then alcohol (lipid removal), thereby obtaining a chitin material (crab shell chitin material). Three hCBD-BAF proteins (hCBD-HRV3Cs-eBAF-Ym3, hCBD-HRV3Cs-eBAF-R₃, and hCBD-HRV3Cs-eBAF-R₄) were applied to different areas of the obtained chitin material (FIG. 9A), and dried. After storage at room temperature for three days, the chitin material was irradiated with a 505-nm green LED illuminator, and light passing through an orange filter was photographed with a digital camera. FIG. 9B (b-1) shows a bright field image under a fluorescent lamp, and (b-2) shows a fluorescence image. FIGS. 9B (b-1) and (b-2) show photographs taken from the same angle. The green color in the negative control and the uncoated part is caused by the reflection of the irradiating green light. Further, FIG. 9B (b-3) shows a fluorescence image of the same sample after dry storage at room temperature for 10 months.

Spectral Measurement of BAFs Used in hCBD-BAF

The spectrum of each of the BAF proteins (eBAF-Ym3, eBAF-R₃, and eBAF-R₄) used, respectively, in the three hCBD-BAF proteins (hCBD-HRV3Cs-eBAF-Ym3, hCBD-HRV3Cs-eBAF-R₃, and hCBD-HRV3Cs-eBAF-R₄) was measured according to the method described in PTL 2. FIGS. 11 to 13 show the results.

### Binding of CBD-BAF Protein to Cicada Exuvia, and Luminescence Observation

A cicada exuvia was used as a chitin material. hCBD-HRV3Cs-eBAF-Ym3 was directly applied to the cicada exuvia. This hybrid material was immersed in the above-mentioned reaction buffer, and a luciferin solution was added. Then, the luminescence state was recorded with a digital camera. FIG. 14 shows the results.

1. A chimeric protein comprising a luminescent domain and a cellulose- and/or chitin-binding domain, the luminescent domain comprising at least one luminescent protein selected from the group consisting of luciferases and fluorescent proteins.

2. The chimeric protein according to claim 1, wherein the luminescent domain is bound to the cellulose- and/or chitin-binding domain directly or via a first linker.

3. The chimeric protein according to claim 1, wherein the luminescent domain comprises a luciferase and a fluorescent protein, and energy transfer (BRET) from the luciferase to the fluorescent protein can occur.

4. The chimeric protein according to claim 3, wherein the luciferase is bound to the fluorescent protein via a second linker.

5. The chimeric protein according to claim 1, wherein the fluorescent protein is GFP, YFP, BFP, CFP, OFP, DsRED, or RFP.

6. The chimeric protein according to claim 5, wherein the fluorescent protein is YFP or RFP.

7. The chimeric protein according to claim 1, wherein the first linker and/or the second linker comprises a protease cleavage sequence.

8. DNA that encodes the chimeric protein according to claim 1, or its complementary strand.

9. A luminescent material wherein the chimeric protein according to claim 1 is bound to a cellulose- or chitin-comprising granule, bead, sheet, or film.

10. The luminescent material according to claim 9, wherein the material is in dried form.

11. The chimeric protein according to claim 2, wherein the luminescent domain comprises a luciferase and a fluorescent protein, and energy transfer (BRET) from the luciferase to the fluorescent protein can occur.

12. The chimeric protein according to claim 11, wherein the luciferase is bound to the fluorescent protein via a second linker.

13. The chimeric protein according to claim 12, wherein the fluorescent protein is GFP, YFP, BFP, CFP, OFP, DsRED, or RFP.

14. The chimeric protein according to claim 13, wherein the fluorescent protein is YFP or RFP.

15. The chimeric protein according to claim 14, wherein the first linker and/or the second linker comprises a protease cleavage sequence.

16. DNA that encodes the chimeric protein according to claim 15, or its complementary strand.

17. A luminescent material, wherein the chimeric protein according to claim 15 is bound to a cellulose- or chitin-comprising granule, bead, sheet, or film.

18. The luminescent material according to claim 17, wherein the material is in dried form.
